(19) United States
(12) Patent Application Publication
(10) Pub. No.: US 2023/0191122 A1
(43) Pub. Date: Jun. 22, 2023

(54) SELF-DRIVING PULSE MICROCURRENT COSMETIC MASK

(71) Applicants: Jue WANG, Beijing (JP); JIAXING JUECHUANG TECHNOLOGY CO., LTD., Jiaxing (JP); YANGTZE RIVER DELTA (JIAXING) NANO APPLIED TECHNOLOGY RESEARCH INSTITUTE, Jiaxing (JP)

(72) Inventors: Jue WANG, Beijing (CN); Xia CAO, Beijing (CN)

(73) Assignees: Jue WANG, Beijing (JP); JIAXING JUECHUANG TECHNOLOGY CO., LTD., Jiaxing (JP); YANGTZE RIVER DELTA (JIAXING) NANO APPLIED TECHNOLOGY RESEARCH INSTITUTE, Jiaxing (JP)

(21) Appl. No.: 18/113,090

(22) Filed: Feb. 23, 2023

Related U.S. Application Data
(63) Continuation-in-part of application No. PCT/CN2021/113138, filed on Aug. 18, 2021.

(30) Foreign Application Priority Data
Aug. 28, 2020 (CN) 202010881998.6
Jun. 15, 2021 (CN) 202121325961.1

(51) Int. Cl. A61N 1/32; A61N 1/36; A45D 44/00

(52) U.S. Cl. CPC A61N 1/328; A61N 1/36014; A45D 44/002

(57) ABSTRACT

A self-driving pulse microcurrent cosmetic mask is provided. The self-driving pulse microcurrent cosmetic mask includes not less than one layer of the mask body. It can work by rubbing or massaging the cosmetic mask to generate Maxwell's displacement electric field and pulse microcurrent, which stimulate the expansion of blood vessels from point to plane on the skin, improve the circulation of blood and lymphatic fluid, accelerate cell metabolism and enhance skin vitality; open the gap between skin cells and promote the absorption of nutrients; clean the skin and kill bacteria and mites on the skin, etc., thus achieving effects of anti-aging, skin tightening and skin whitening and delicate. The cosmetic mask does not need any power source, is easy to carry and operate, and is thus suitable for many parts of the body and people of all ages.

### CROSS REFERENCE TO THE RELATED APPLICATIONS

This application is a continuation-in-part application of International Application No. PCT/CN2021/113138, filed on Aug. 18, 2021, which is based upon and claims priority to Chinese Patent Application No. 202010881998.6, filed on Aug. 28, 2020; and Chinese Patent Application No. 202121325961.1, filed on Jun. 15, 2021, the entire contents of which are incorporated herein by reference.

### TECHNICAL FIELD

The present invention relates to the field of cosmetic technologies, in particular to a self-driving pulse microcurrent cosmetic mask.

### BACKGROUND

With the development of technology, cosmetic technology is also constantly innovating, and research has found that the use of microcurrent can get anti-aging, skin tightening and other effects, the microcurrent cosmetic instrument, microcurrent mask and other products based on this principle are popular. However, most microcurrent cosmetic devices on the market require periodic charging, which greatly limits their convenience, and such products currently on the market are expensive and unsuitable for popularisation among the general public.

The present invention provides a self-driving pulse microcurrent cosmetic mask which is simple to operate, easy to carry, does not require any power source, and does not have the hidden danger of excessive current. It can be disassembled and washed for repeated use, and is suitable for people of all ages. This is a brand new technology application, applying our new cutting-edge technology to the field of beauty and skincare, so that we can protect our skin anytime and anywhere to facilitate our life and make us more beautiful and healthy.

### SUMMARY

The technical problem to be solved by the present invention is to provide a self-driving pulse microcurrent cosmetic mask. It includes not less than one layer of the mask body. The cosmetic mask can be applied to the skin without an external power supply. It can work by rubbing or massaging the cosmetic mask to generate Maxwell's displacement electric field and Maxwell's displacement current, which achieve stimulation of the skin from point to plane, avoiding local skin damage and allergy and making the skin delicate and revitalized.

The cosmetic mask in this new technology can generate a short-circuit current of up to 50 microamps and an open-circuit voltage of up to 2200-2400 volts. Using the cosmetic mask made from special materials in this technology, the mask body generates a pulse electric field and a pulse current that can light up a 1-watt light bulb. The microcurrent cosmetic mask provided by the present invention works on the joint action of Maxwell's displacement electric field and the point current, which has a more uniform stimulation of the skin than the action of the point current alone on the market, avoiding local skin allergy and damage.

The self-driving pulse cosmetic mask consists of one layer of the mask body. The cosmetic mask can be applied to the skin. It can work by rubbing or massaging the cosmetic mask to generate Maxwell's displacement electric field and pulse microcurrent, which act on the skin surface without damage to the human body, but have a stimulating effect on the skin and act as a treatment.

The self-driving pulse cosmetic mask consists of one layer of the mask body, and the mask body has the massage bodies with a strong triboelectric performance on the surface or inside. The cosmetic mask can be applied to the skin. It can work by rubbing or massaging the cosmetic mask to generate Maxwell's displacement electric field and pulse microcurrent, which acts on the skin surface to achieve a continuous and gentle treatment of the skin with some anti-inflammatory and anti-bacterial effects.

The self-driving pulse cosmetic mask consists of two or more layers of the mask body. The cosmetic mask is applied to the skin and works through rubbing or massaging on the cosmetic mask. The layers are constantly in contact or misaligned with each other, generating Maxwell's displacement electric field and pulse microcurrent that act on the skin surface and provide effective stimulation to human skin.

The self-driving pulse cosmetic mask consists of two or more layers of the mask body, and there are movable intermediates between layers. The cosmetic mask is applied to the skin and works through rubbing or massaging on the cosmetic mask. The mutual friction or collision between the movable intermediates, between the movable intermediates and the layer, generates Maxwell's displacement electric field and pulse microcurrent, which acts on the skin surface by stimulating blood circulation and energizing the skin.

The structure of the movable intermediates is unlimited and can be granular, tubular, fragmented, mesh, flocculent, spherical, rod-like, including but not limited to nanowires, nanotubes, nanoparticles, nanorods, nanoflowers, nanogrooves, micrometer grooves, nanocones, micrometer cones, nanospheres, and micrometer spherical structures.

The self-driving pulse cosmetic mask includes two parts: a mask body and a cosmetic brush. The mask body and the cosmetic brush are respectively independent devices with complete structures. The cosmetic brush includes a base and a contact surface. The cosmetic brush and the mask body are in constant contact or friction under the action of force, generating Maxwell's displacement electric field and pulse microcurrent to stimulate skin blood circulation and activate the skin vitality.

The cosmetic mask can be used anytime, anywhere, with or without make-up, on dry or wet surfaces. It is green, safe and environmentally friendly.

The self-driving pulse microcurrent cosmetic mask cannot have a cosmetic brush, so the hand interacts directly with the mask body to generate pulse microcurrent, stimulating the skin's blood circulation and rejuvenating the skin.

The self-driving pulse microcurrent cosmetic mask does not need to be connected to an external power source, there is no safety risk of leakage and there is little irritation and damage to the skin.

The self-driving pulse microcurrent cosmetic masks can be applied to all parts of the body, mainly including: face, chest, hands, legs, neck, feet, etc., especially to stimulate mammary development, tighten and lift the skin, and reduce neck lines and fine lines.

The mask body is manufactured from flexible or rigid materials, mainly including textile materials, silk materials, cotton materials, chemical fiber materials, polymer materials, organic materials, inorganic materials, and organic-inorganic composites. The size and shape of the mask body can be adjusted and changed according to the area of use.

The mask body can be a single-layer membrane or a multi-layer membrane, and the mask body can be a mesh structure, a dense structure, or a porous structure.

The structure of the surface of the cosmetic brush in contact with the mask body can be made bristle-like, a flat membrane, or a porous structure.

The inner surface of the mask body is in contact with the skin. Through the interaction between the mask body and the cosmetic brush or the hand, the pulse microcurrent is generated by the mask body to stimulate blood circulation in the skin.

The effect of the self-driving pulse microcurrent cosmetic mask is related to the contact force, contact time and contact area of the mask body and the cosmetic brush or hand. The contact force and time are determined according to the individual skin's ability to withstand. The force can be applied vertically, horizontally, or in an inclined direction. The forces include friction, contact, collision, or flapping.

The collision, friction or contact of any matter can generate an electromagnetic field and a displacement current. It is a new discovery from 0 to 1. The human body has electrical conductivity, so Maxwell's displacement electric field and pulse microcurrent generated by the self-driving pulse cosmetic mask can act on the skin surface and promote skin blood circulation, which plays an effective stimulation of human skin, to achieve the activation of cells, skin tightening, anti-aging, promote breast development and hair growth and other care effects.

In addition to improving the condition of the skin, the self-driving pulse microcurrent cosmetic mask can also kill and remove bacteria, mites and other microorganisms from the skin, leaving the skin clean, fresh and revitalized.

The color of the self-driving pulse microcurrent cosmetic mask can be changed to suit the client's requirements.

The mask is adapted to other parts of the face by changing its shape, keeping other structures intact, to make one or more of the cosmetic patches for the nasolabial folds, forehead, nose, neck, lips and plump cheeks.

### BRIEF DESCRIPTION OF THE DRAWINGS

FIG. 1 is a schematic diagram of a self-driving pulse microcurrent cosmetic mask according to the present invention;

FIG. 2 is a schematic diagram of a self-driving pulse microcurrent cosmetic mask according to the present invention;

FIG. 3 is a schematic diagram of a self-driving pulse microcurrent cosmetic mask according to the present invention;

FIG. 4 is a schematic diagram of a self-driving pulse microcurrent cosmetic mask according to the present invention;

FIG. 5 is a principle diagram of a self-driving pulse microcurrent cosmetic mask according to the present invention;

FIG. 6 is a principle diagram of a self-driving pulse microcurrent cosmetic mask according to the present invention;

FIG. 7 is a schematic diagram of the open circuit voltage generated by a self-driving pulse microcurrent cosmetic mask;

FIG. 8 is a schematic diagram of the short-circuit current generated by a self-driving pulse microcurrent cosmetic mask;

FIG. 9 is a schematic diagram of a light bulb powered by a self-driving pulse microcurrent cosmetic mask;

FIG. 10 is a schematic diagram of a self-driving pulse microcurrent cosmetic mask according to embodiment 1 of the present invention;

FIG. 11 is a schematic diagram of a self-driving pulse microcurrent cosmetic mask according to embodiment 2 of the present invention;

FIG. 12 is a schematic diagram of a self-driving pulse microcurrent cosmetic mask according to embodiment 3 of the present invention;

FIG. 13 is a schematic diagram of a self-driving pulse microcurrent cosmetic mask according to embodiment 4 of the present invention;

FIG. 14 is a schematic diagram of a self-driving pulse microcurrent cosmetic mask according to embodiment 5 of the present invention.

### IN THE DRAWINGS

1 Base;

2 Contact surface;

3 Mask body;

4 Cosmetic brush;

5 Outer surface of the mask body;

6 Inner surface of the mask body;

7 Movable intermediate;

8 Massage body;

9 Hand;

10 Skin;

11 Mask type;

12 Eye film type;

13 Hand film or glove type;

14 Leg film type;

15 Leg;

16 Particles;

17 Breast patch type.

### DETAILED DESCRIPTION OF THE EMBODIMENTS

For better clarity of the technical problem to be solved by the present invention as well as the technical solutions and advantages of the present invention, a detailed description will be given below with reference to the accompanying drawings and specific embodiments.

The present invention provides a self-driving pulse microcurrent cosmetic mask.

The self-driving pulse microcurrent cosmetic mask includes not less than one layer of the mask body. The cosmetic mask can be applied to the skin without an external power supply. It can work by rubbing or massaging the cosmetic mask, and generating Maxwell's displacement electric field and pulse microcurrent, which stimulate the expansion of blood vessels from point to plane on the skin, improve the circulation of blood and lymphatic fluid, accelerate cell metabolism and enhance skin vitality; open the gap between skin cells and promote the absorption of nutrients; clean the skin and kill bacteria and mites on the skin, etc., thus achieving effects of anti-aging, skin tightening and skin whitening and delicate.

The cosmetic mask is easy to carry, easy to operate, which can be used anytime, anywhere, with or without make-up, on dry or wet surfaces. It is green, safe and environmentally friendly.

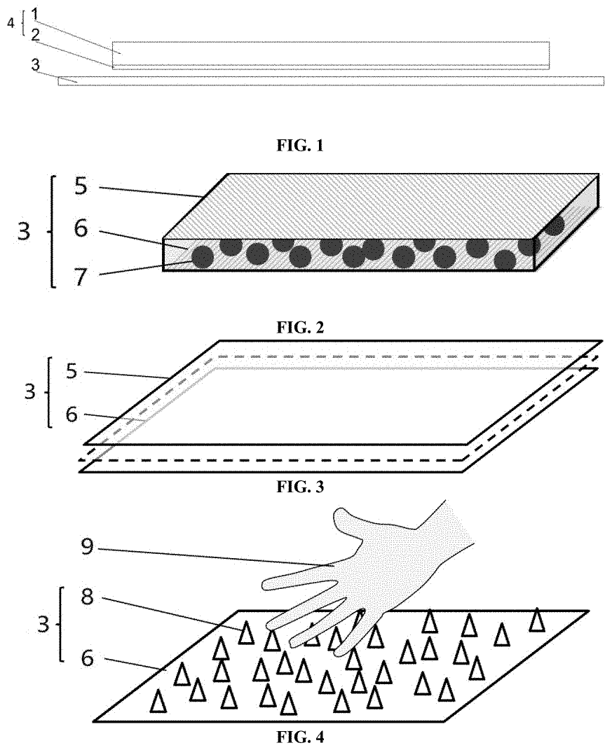

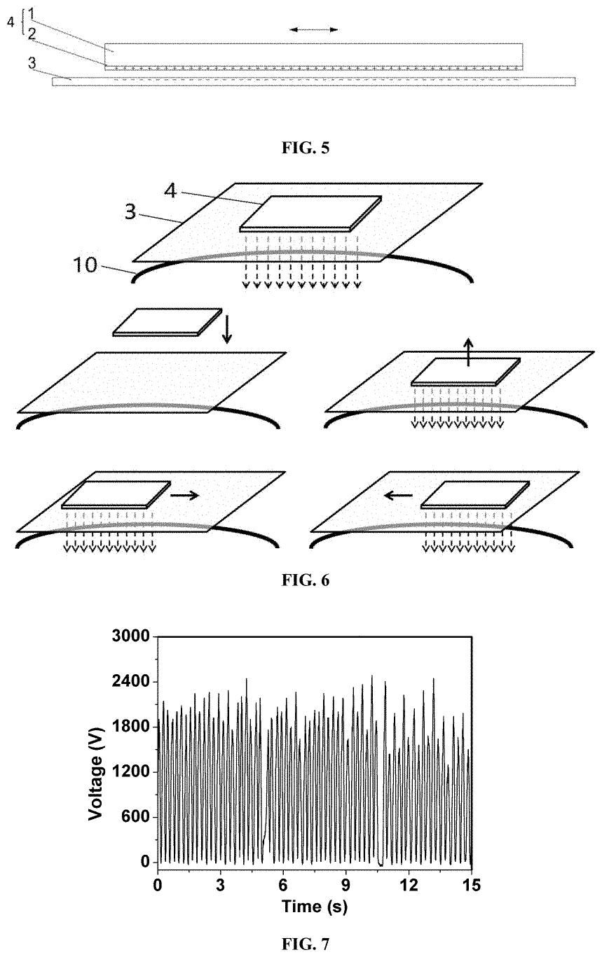

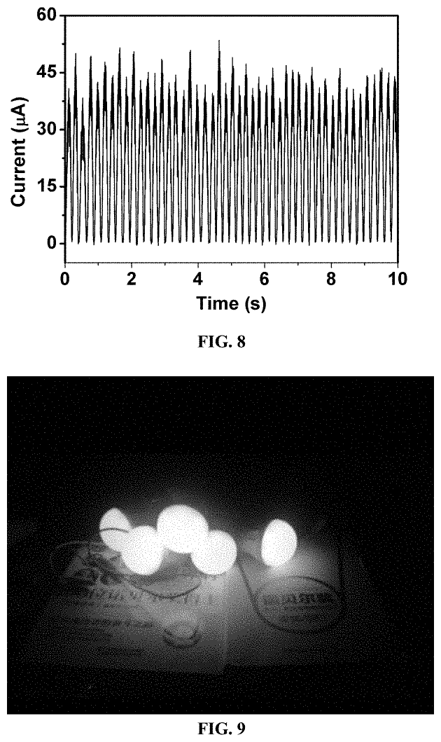

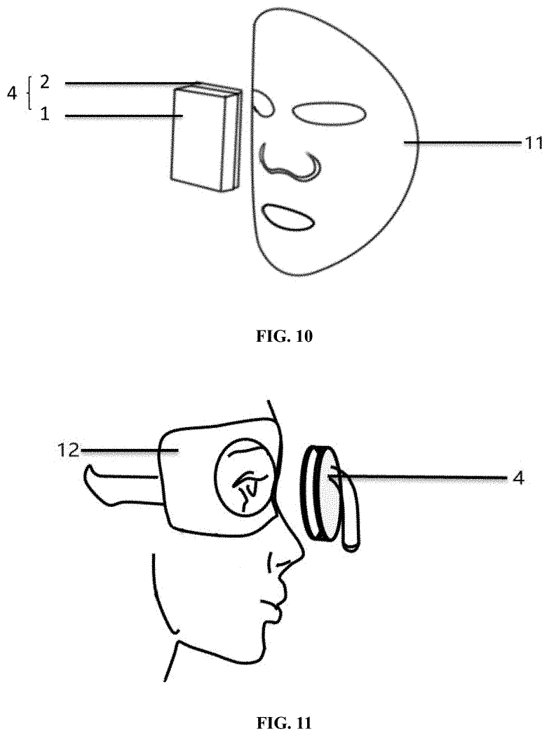

As shown in FIG. 1, FIG. 2, FIG. 3, FIG. 4, FIG. 5, FIG. 6, the schematic diagram and the principle diagram of the cosmetic mask are shown respectively. The cosmetic mask is different from the microcurrent cosmetic devices in the current market. The cosmetic mask works on a joint action of Maxwell's displacement electric field and the pulse microcurrent, which has a more uniform and continuous stimulation of the skin than the action of the point current alone, avoiding local skin damage and allergy.

As shown in FIG. 1, the cosmetic mask consists of a mask body (3) and a cosmetic brush (4). The cosmetic brush includes a base (1) and a contact surface (2). The cosmetic brush and the mask body are in constant contact or friction under the action of force, generating Maxwell's displacement electric field and pulse microcurrent to stimulate skin blood circulation and activate the skin vitality.

As shown in FIG. 2, the cosmetic mask consists of two or more layers of the mask body (5, 6), and there are movable intermediates between layers. The cosmetic mask is applied to the skin and works through directly massaging on the cosmetic mask by hand. The mutual friction or collision between the movable intermediates, between the movable intermediates and the mask body, generates Maxwell's displacement electric field and pulse microcurrent to act on the skin surface. The structure of the movable intermediates is unlimited and can be granular, tubular, fragmented, mesh, flocculent, spherical, rod-like.

As shown in FIG. 3, the cosmetic mask consists of two or more layers of the mask body (5, 6). The cosmetic mask is applied to the skin and works through rubbing or massaging on the cosmetic mask. The layers are constantly in contact or misaligned with each other, generating Maxwell's displacement electric field and pulse microcurrent that act on the skin surface and provide effective stimulation to human skin.

As shown in FIG. 4, the self-driving pulse cosmetic mask consists of one layer of the mask body, and the mask body has the massage bodies (8) with a strong triboelectric performance on the surface. The cosmetic mask can be attached to the skin. It can work by the movement of the human hand (9) to make the cosmetic mask generate Maxwell's displacement electric field and pulse microcurrent, which acts on the skin surface to achieve a continuous and gentle treatment of the skin. The structure of the massage bodies is unlimited and can be granular, tubular, fragmented, mesh, flocculent, spherical, or rod-like.

FIG. 5 and FIG. 6 are principle diagrams of the cosmetic mask. The cosmetic mask can be applied to the skin (10). The mask body is in contact or approach to the skin, and the human hand or other object interacts with the cosmetic mask through forces that occur with each other. The force can be applied vertically, horizontally, or in an inclined direction. The forces include but are not limited to friction, contact, collision, or flapping. The collision, friction or contact of any matter can generate an electromagnetic field and a displacement current. It is a new discovery from 0 to 1. The Maxwell's displacement electric field and pulse microcurrent generated by the cosmetic mask can act on the skin surface, effectively stimulating the skin from point to plane, and promoting the skin's blood circulation.

As shown in FIG. 7, FIG. 8 and FIG. 9, the cosmetic mask in this new technology can generate a short-circuit current of up to 50 microamps and an open-circuit voltage of up to 2200-2400 volts. Using the cosmetic mask made from special materials in this technology, the mask body generates a pulse electric field and a pulse current that can light up a 1-watt light bulb.

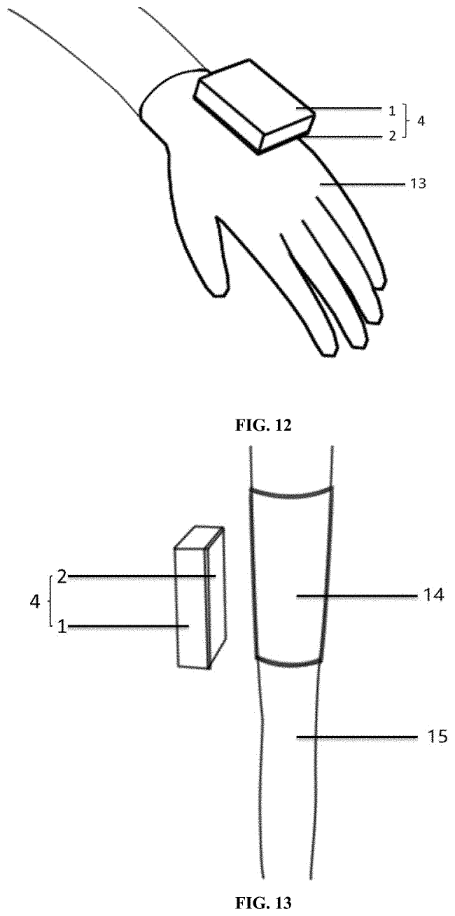

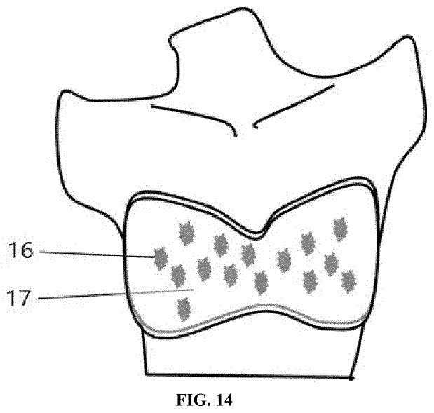

As shown in FIG. 10, FIG. 11, FIG. 12, FIG. 13, FIG. 14, the self-driving pulse microcurrent cosmetic masks can be applied to all parts of the body, mainly including: face, chest, hands, legs, neck, feet, etc., especially to stimulate mammary development, tighten and lift the skin, reduce neck lines and fine lines.

A description is given below in conjunction with the specific embodiments.

### Embodiment 1

As shown in FIG. 10, a self-driving pulse microcurrent cosmetic mask in this embodiment is used for facial skin maintenance. The self-driving pulse microcurrent cosmetic mask is made into a mask type 11, so that it fits the skin of the face. The mask can generate Maxwell displacement electric field and Maxwell displacement current by using a cosmetic brush or other objects to rub or touch on the surface of the mask. It stimulates the blood circulation of the facial skin, shrinks the pores, and makes the skin tight, red and delicate. It can also effectively lighten and fade the lines, headlines, necklines, etc.

### Embodiment 2

As shown in FIG. 11, a self-driving pulse microcurrent cosmetic mask in this embodiment is used for the maintenance of eye skin. The cosmetic mask is made into an eye film type 12, so that it fits the eye skin. The eye film can generate Maxwell displacement electric field and Maxwell displacement current by using a cosmetic brush to rub or touch the surface of the eye film. It can provide continuous, even and gentle stimulation of the eye skin without an external power source, and can tighten the skin, eliminate fine lines and spots, kill mites and bacteria, and stimulate skin cells to rejuvenate.

### Embodiment 3

As shown in FIG. 12, a self-driving pulse microcurrent cosmetic mask in this embodiment is used for the maintenance and care of hand skin. The self-driving pulse microcurrent cosmetic mask is made into a hand film or glove type 13, so that it fits the skin of the hand. The hand film or glove generates Maxwell displacement electric field and Maxwell displacement current by using a cosmetic brush or other objects to rub or touch the surface of the hand film or glove. It stimulates the blood circulation of the hand skin, shrinks the pores and makes the hand skin firm and delicate.

### Embodiment 4

As shown in FIG. 13, a self-driving pulse microcurrent cosmetic mask in this embodiment is used for the maintenance and care of the leg skin. The self-driving pulse microcurrent cosmetic mask is made into a leg film type 14, so that it fits the skin of the human leg 15. The leg film generates Maxwell displacement electric field and Maxwell displacement current by using a cosmetic brush or other objects to rub or touch the surface of the leg film. It can stimulate the blood circulation of the leg skin, shrinks the pores, and makes the leg skin tight and delicate.

### Embodiment 5

As shown in FIG. 14, a self-driving pulse microcurrent cosmetic mask in this embodiment is used for breast skin care. The cosmetic mask is made into a breast patch type 17. The breast patch is composed of two layers of membranes, and there are movable intermediates of particles (16) between the layers. It can work by rubbing or massaging the breast patch with the human hand or other objects. The mutual friction or collision between the movable intermediates, between the movable intermediates and the membranes, generates Maxwell's displacement electric field and pulse microcurrent, which acts on the skin surface to promote blood circulation of the breast, activate cells, promote breast development, tighten and lift, and prevent breast sagging.

What is claimed is:

1. A self-driving pulse microcurrent cosmetic mask, comprising at least one layer of a mask body, the self-driving pulse microcurrent cosmetic mask is configured to be applied to a skin without an external power supply, the self-driving pulse microcurrent cosmetic mask is configured to work by rubbing or massaging to generate Maxwell's displacement electric field and Maxwell's displacement current, wherein the Maxwell's displacement electric field and the Maxwell's displacement current achieve a stimulation of the skin from a point to a plane, avoiding a local skin damage and an allergy and making the skin delicate and revitalized.

2. The self-driving pulse microcurrent cosmetic mask according to claim 1, wherein the self-driving pulse microcurrent cosmetic mask comprises one layer of the mask body, the self-driving pulse microcurrent cosmetic mask is configured to be applied to the skin, the self-driving pulse microcurrent cosmetic mask is configured to work by rubbing or massaging to generate the Maxwell's displacement electric field and a pulse microcurrent, wherein the Maxwell's displacement electric field and the pulse microcurrent act on a skin surface without a damage to a human body, but have a stimulating effect on the skin and act as a treatment.

3. The self-driving pulse microcurrent cosmetic mask according to claim 2, wherein the self-driving pulse microcurrent cosmetic mask comprises the one layer of the mask body, and the one layer of the mask body has massage bodies with a strong triboelectric performance on a surface or inside, the self-driving pulse microcurrent cosmetic mask is configured to be applied to the skin, the self-driving pulse microcurrent cosmetic mask is configured to work by rubbing or massaging to generate the Maxwell's displacement electric field and the pulse microcurrent, wherein the Maxwell's displacement electric field and the pulse microcurrent act on the skin surface to achieve a continuous and gentle treatment of the skin with some anti-inflammatory and anti-bacterial effects.

4. The self-driving pulse microcurrent cosmetic mask according to claim 1, wherein the self-driving pulse microcurrent cosmetic mask comprises at least two layers of the mask body, the self-driving pulse microcurrent cosmetic mask is applied to the skin and works through rubbing or massaging on a cosmetic mask, the layers are constantly in contact or misaligned with each other, generating the Maxwell's displacement electric field and a pulse microcurrent, wherein the Maxwell's displacement electric field and the pulse microcurrent act on a skin surface and provide an effective stimulation to a human skin.

5. The self-driving pulse microcurrent cosmetic mask according to claim 4, wherein the self-driving pulse microcurrent cosmetic mask comprises the at least two layers of the mask body, and movable intermediates are configured between the layers, the self-driving pulse microcurrent cosmetic mask is applied to the skin and works through rubbing or massaging on the cosmetic mask, a mutual friction or collision between the movable intermediates, between the movable intermediates and the layer, generates the Maxwell's displacement electric field and the pulse microcurrent, wherein the Maxwell's displacement electric field and the pulse microcurrent act on the skin surface by stimulating a blood circulation and energizing the skin.

6. The self-driving pulse microcurrent cosmetic mask according to claim 1, wherein the self-driving pulse microcurrent cosmetic mask comprises a mask body and a cosmetic brush, the mask body and the cosmetic brush are respectively independent devices with complete structures, the cosmetic brush and the mask body are in constant contact or friction under an action of a force, generating the Maxwell's displacement electric field and a pulse microcurrent to stimulate a skin blood circulation and activate a skin vitality.

7. The self-driving pulse microcurrent cosmetic mask according to claim 6, wherein the cosmetic brush comprises a base and a contact surface with a bristle-like, flat film or porous structure.

8. The self-driving pulse microcurrent cosmetic mask according to claim 1, wherein the self-driving pulse microcurrent cosmetic mask is applied to all parts of a body, comprising: a face, a chest, hands, legs, a neck and feet, stimulating a blood circulation and firming and lifting the skin.

9. The self-driving pulse microcurrent cosmetic mask according to claim 8, wherein the self-driving pulse microcurrent cosmetic mask is adapted to other parts of the face by changing a shape of the self-driving pulse microcurrent cosmetic mask and keeping other structures intact, to make at least one of cosmetic patches for nasolabial folds, a forehead, a nose, a neck, lips and plump cheeks.

10. The self-driving pulse microcurrent cosmetic mask according to claim 1, wherein the at least one layer of the mask body is made of one of flexible, rigid and rigid-flexible materials and a size and shape of the at least one layer of the mask body is configured to be adapted to an area of use.

11. The self-driving pulse microcurrent cosmetic mask according to claim 1, wherein an inner surface of the at least one layer of the mask body interacts with the skin under a force comprising friction, contact, collision or tapping, through an interaction between the at least one layer of the mask body and a cosmetic brush or a hand, a pulse microcurrent is generated by the at least one layer of the mask body to stimulate a blood circulation in the skin.
